(19) United States
(12) Patent Application Publication
(10) Pub. No.: US 2024/0368681 A1
(43) Pub. Date: Nov. 7, 2024

(54) QUANTITATIVE PCR METHOD USING INTERNAL CONTROL

(71) Applicants: SHIMADZU CORPORATION, Kyoto (JP); NIHON TECHNO SERVICE CO., LTD., Ibaraki (JP)

(72) Inventors: Masamitsu SHIKATA, Kyoto-shi, Kyoto (JP); Yasuhiro TOMARU, Tsukuba-shi, Ibaraki (JP)

(73) Assignees: SHIMADZU CORPORATION, Kyoto (JP); NIHON TECHNO SERVICE CO., LTD., Ibaraki (JP)

(21) Appl. No.: 18/685,768

(22) PCT Filed: Aug. 23, 2022
(86) PCT No.: PCT/JP2022/031737

§ 371 Date: Feb. 22, 2024

(30) Foreign Application Priority Data
Aug. 27, 2021 (JP) 2021-138596

(51) Int. Cl. C12Q 1/6851

(52) U.S. Cl. CPC C12Q 1/6851

(57) ABSTRACT

A method for quantifying DNA in a specimen includes: a step of performing PCR by adding, to a first container containing a specimen and a second container containing a known amount of standard DNA, the same number of copies of internal control DNA, a PCR primer pair for amplifying the DNA in the specimen and the standard DNA, a PCR primer pair for amplifying the internal control DNA, an oligonucleotide fluorescence-labeled probe, an oligonucleotide fluorescence-labeled probe, and a PCR buffer solution containing a DNA polymerase; a step of measuring a difference between Ct values in the PCR by comparing Ct values for the internal control DNA contained in the first container and the second container; a step of correcting a Ct value for the DNA in the specimen contained in the first container; and a step of measuring an amount of DNA in the specimen from a calibration curve.

### TECHNICAL FIELD

The present invention relates to a method for quantify ing DNA using PCR and a kit for performing the method.

### BACKGROUND ART

Target DNA in a specimen can be quantified by amplifying it by PCR (polymerase chain reaction). One of the methods for quantifying the target DNA is a real-time PCR method. In this method, a dilution series of standard DNA whose number of copies is known is prepared, and while these are amplified by PCR, the amount of the amplification product is detected in real time to prepare a calibration curve. The calibration curve can be prepared, for example, by plotting the Ct (threshold cycle) value of each diluted solution on a graph in which the vertical axis represents the number of copies of standard DNA contained in each diluted solution of the dilution series and the horizontal axis represents the Ct value. The number of copies of the target DNA in the specimen can be determined by determining the Ct value for the target DNA and applying the Ct value to the calibration curve. The dilution series of standard DNA is prepared using a container different from the container for preparing the specimen. In the real-time PCR method, a well of a PCR plate or a PCR tube is usually used as these containers.

### SUMMARY OF INVENTION

### Technical Problem

PCR is usually performed using DNA extracted from a specimen and purified. However, the operation of extracting and purifying DNA from a specimen is complicated and takes a long time. In contrast, in direct PCR, when the detection target is DNA, the DNA is released by chemical treatment of a specimen or by dissolving the specimen in a thermal denaturation step in PCR. In direct PCR, when the detection target is RNA. RNA is released by chemical treatment or heat treatment of a specimen, and complementary DNA converted from RNA by a reverse transcription reaction is amplified directly from the specimen. Therefore, direct PCR is easy to operate, and the operation time is also shortened.

However, when target DNA in the specimen is quantified by direct PCR using the calibration curve, a PCR reaction solution containing the specimen contains a component derived from the specimen, for example, a PCR-inhibiting substance or the like, but the PCR reaction solution containing standard DNA for preparing the calibration curve does not contain such a component derived from the specimen, and therefore PCR is performed in a state in which a difference occurs in the composition of the PCR reaction solution. In addition, the degree to which PCR is affected by the PCR-inhibiting substance or the like varies depending on the specimen, which may cause differences in the progress of PCR between wells. For this reason, there is a possibility that the signal value obtained from the real-time PCR method is affected by the difference in the composition of the PCR reaction solution and/or the difference between the specimens in the PCR process, but there is no means for confirming whether or not the amount of target DNA present in the specimen is accurately measured on the basis of the calibration curve.

An object of the present invention is to provide a method for more accurately quantifying target DNA in a specimen and a kit for performing the method, the method including detecting an error in a signal value and correcting the detected error, by measuring an influence on the signal value obtained from a real-time PCR method, the influence due to a composition difference of reaction solutions between a PCR reaction solution containing standard DNA for preparing a calibration curve and a PCR reaction solution containing target DNA in the specimen and/or a difference in specimens in a PCR process.

### Solution to Problem

That is, the object of the present invention is achieved by the following invention.
[1] A method for quantifying DNA in a specimen, the method comprising:
a step of performing PCR by adding, to a first container containing a specimen and a second container containing a known amount of standard DNA, respectively a same number of copies of internal control DNA, a PCR primer pair for amplifying the DNA in the specimen and the standard DNA, a PCR primer pair for amplifying the internal control DNA, an oligonucleotide fluorescence-labeled probe for detecting the DNA in the specimen and the standard DNA, an oligonucleotide fluorescence-labeled probe for detecting the internal control DNA, and a PCR buffer solution containing a DNA polymerase;
a step of measuring a difference between Ct values in the PCR by comparing Ct values for the internal control DNA contained in the first container and the second container;
a step of correcting a Ct value for the DNA in the specimen contained in the first container on a basis of the difference between the Ct values; and
a step of measuring an amount of the DNA in the specimen from a calibration curve prepared on a basis of the amount of the standard DNA contained in the second container and the corrected Ct value.
[2] The method according to [1], wherein the specimen is a sample selected from the group consisting of an organism sample, an organism-derived sample, an environmental sample, and an environmental-derived sample.
[3] The method according to [1], wherein the specimen is a sample selected from the group consisting of an excrement sample, an excrement-derived sample, a vomit sample, and a vomit-derived sample.
[4] The method according to any one of [1] to [3], wherein the specimen contains a pathogen.
[5] The method according to [4], wherein the pathogen is a virus, a bacterium, a fungus, or a protozoan.
[6] The method according to any one of [1] to [5], wherein the internal control DNA has a chain length of 50 to 200 bp and a GC-content of 40 to 60%.
[7] The method according to any one of [1] to [6], wherein the PCR buffer solution contains a surfactant.
[8] The method according to [7], wherein the surfactant is a nonionic surfactant.
[9] The method according to any one of [1] to [8], wherein the PCR buffer solution is a tris buffer solution containing KCl, MgCl₂, and dNTP mix (mixture consisting of dATP, dGTP, dCTP, and dTTP).
[10] The method according to any one of [1] to [9], wherein the PCR buffer solution contains a substance that is bound to PCR-inhibiting substances, i.e., a bio-derived negative charge substance adsorbed to DNA polymerase and a bio-derived positive charge substance adsorbed to DNA, and neutralizes a PCR inhibitory action of the negative charge substance and the positive charge substance.
[11] A kit for quantifying DNA in a specimen, the kit comprising:
internal control DNA;
a PCR primer pair for amplifying the DNA in the specimen and standard DNA;
a PCR primer pair for amplifying the internal control DNA;
an oligonucleotide fluorescence-labeled probe for detecting the DNA in the specimen and the standard DNA;
an oligonucleotide fluorescence-labeled probe for detecting the internal control DNA;
a DNA polymerase; and
a PCR buffer solution.
[12] The kit according to [11], wherein the internal control DNA has a chain length of 50 to 200 bp and a GC-content of 40 to 60%.
[13] The kit according to [11] or [12], wherein the PCR buffer solution contains a surfactant.
[14] The kit according to [13], wherein the surfactant is a nonionic surfactant.
[15] The kit according to any one of [11] to [14], wherein the PCR buffer solution is a tris buffer solution containing KCl, MgCl₂, and dNTP mix (mixture consisting of dATP, dGTP, dCTP, and dTTP).
[16] A kit according to any one of [11] to [15], wherein the PCR buffer solution contains a substance that is bound to PCR-inhibiting substances, i.e., a bio-derived negative charge substance adsorbed to DNA polymerase and a bio-derived positive charge substance adsorbed to DNA, and neutralizes a PCR inhibitory action of the negative charge substance and the positive charge substance.

### Advantageous Effects of Invention

Since the real-time PCR method is performed by adding, to a first container containing a specimen and a second container for creating a calibration curve containing a known amount of DNA, respectively the same number of copies of internal control DNA, even when a positive or negative error occurs in a signal value obtained from the real-time PCR method due to a composition difference of reaction solutions between a PCR reaction solution for a specimen and a PCR reaction solution for preparing a calibration curve and/or a difference between specimens in a PCR process, it is possible to detect the error by comparing amplification curves for the internal control DNA contained in the first container and the second container. Furthermore, since the amount of target DNA in the specimen is measured on the basis of the detected error, it is possible to more accurately quantify the target DNA.

### DESCRIPTION OF EMBODIMENTS

### [Specimen]

Examples of the specimen in the present invention include an organism sample, an organism-derived sample, an environmental sample, and an environmental-derived sample. The organism sample encompasses disruptions and extracts of cells, tissues and/or organs. Examples of the tissues or organs include the brain, spinal cord, bone marrow, conjunctiva, cornea, vitreous, heart, mitral valve, tricuspid valve, lung, pleura, liver, spleen, peritoneum, intestine, lymph nodes, skin, and the like. The organism sample further encompasses blood, including whole blood, plasma, serum, and the like, as well as blood-related samples, lymphatic fluid, saliva, nasal secretions, pharyngeal swab, nasal swab, sweat, tears, tissue fluids (intertissue fluid, intercellular fluid, and interstitial fluid), body cavity fluids (ascites, pleural effusion, pericardial fluid, cerebrospinal fluid, joint fluid, and aqueous humor), pleural, peritoneal, cranial or intraspinal exudates (such as pleural or ascitic fluid). The organism sample may be centrifuged, and a supernatant or a centrifugal sediment obtained by centrifugation may be used as a specimen. The organism sample encompasses a mixture of the organism sample with a culture solution, a buffer solution, a specimen storage solution, and the like. The buffer solution is not particularly limited, and examples thereof include phosphate buffer solutions, tris buffer solutions, borate buffer solutions, and Good buffer solutions such as HEPES. The organism-derived sample encompasses an organism sample processed by sonication or the like. The environmental sample encompasses any kind of samples including air, soil, dust, water, and the like. The environmental-derived sample encompasses an environmental sample processed by sonication or the like.

As another embodiment of the present invention, examples of the specimen include an excrement sample, an excrement-derived sample, a vomit sample, and a vomit-derived sample. The excrement sample and the vomit sample can be used as specimens as they are, but may be diluted or suspended with distilled water, physiological saline, or a buffer solution. The buffer solution is not particularly limited, and examples thereof include phosphate buffer solutions, tris buffer solutions, borate buffer solutions, and Good buffer solutions such as HEPES. The suspension of the sample may be centrifuged, and the centrifuged supernatant may be used as the specimen. The excrement-derived sample and the vomit-derived sample encompass a wiped sample. The wiped sample is obtained by wiping a finger, a dish, a cooking facility, a toilet facility, a house facility, or the like with a cotton swab, cut cotton, or the like and eluting the wiped substance into a phosphate buffer solution or the like.

The specimen in the present invention may contain a pathogen. Examples of the pathogen include viruses, bacteria, fungi, and protozoa. The viruses encompass DNA viruses and RNA viruses. The DNA viruses are viruses with DNA as a genome and examples thereof include, but are not limited to, Herpes simplex virus type 1 (HSV-1), Herpes simplex virus type 2 (HSV-2), Varicella zoster virus (VZV), Epstein-Barr virus (EBV), Cytomegalovirus (CMV), Human Herpes virus type 6 (HHV-6), Adenovirus, and Papillomavirus. The RNA viruses are viruses having RNA as a genome and examples thereof include, but are not limited to, an enveloped virus with a membrane composed of a lipid bilayer, such as a coronavirus, a human immunodeficiency virus, a hepatitis C virus, a Japanese encephalitis virus, and a dengue virus, and a non-enveloped virus, such as a norovirus, a rotavirus, and a rhinovirus. The genomic RNA of an RNA virus becomes “DNA in a specimen” by generating cDNA by a reverse transcription reaction. Examples of the bacteria include, but are not limited to, Staphylococcus aureus, Chlamydia, Salmonella, Bacillus cereus, Vibrio enteritis, Enterohemorrhagic E. coli O 157, Treponema pallidum, and the like, and the protozoa include, but are not limited to, Toxoplasma and Entamoeba dysenteriae, and the like. A specific base sequence in “DNA in a specimen” can be amplified so as to be detected by selecting a primer pair (forward and reverse) used for amplification of a target gene region in PCR

The standard DNA used in the present invention is not particularly limited as long as it is a DNA having the sequence of the target DNA in the specimen, but it is preferable to approximate the shape and size of the target DNA in order to match the PCR amplification efficiency as much as possible.

### [Real-Time PCR Method]

### (Containers)

A first container and a second container are preferably wells of a PCR plate or PCR tubes. The PCR plates of, for example, 96-well and 386-well formats can be used. As the PCR tube, a single tube having a dose size of about 0.1 to 0.5 mL and a 2 to 12 continuous strip tube can be used. As a material of the first and second containers, polypropylene or the like can be used. The tube color is preferably non-colored or white.

### (PCR Buffer Solution)

The PCR buffer solution contains KCl, MgCl₂, and dNTP mix (deoxyribonucleotide 5′-triphosphate, a mixture consisting of dATP, dGTP, dCTP, and dTTP). The PCR buffer solution is preferably, but not limited to, a tris hydrochloric buffer solution. For dNTP, MgCl₂, KCl, and buffer solutions, appropriate concentrations can be set by those skilled in the art. For example, the concentration of MgCl₂ is 1.5 mM, the concentration of KCl is 35 mM, the concentration of each component in dNTP is 200 μM, and the concentration of tris hydrochloric acid is 10 mM. In one embodiment of the present invention, the PCR buffer solution contains a substance that is bound to PCR-inhibiting substances, i.e., a bio-derived negative charge substance (for example, certain kinds of sugars, dyes, and the like) adsorbed to DNA polymerase and a bio-derived positive charge substance (for example, certain kinds of proteins and the like) adsorbed to DNA, and that neutralizes the PCR inhibitory action of the negative charge substance and the positive charge substance. As the PCR buffer solution, a reagent for gene amplification Ampdirect Plus (registered trademark, Shimadzu Corporation) can be used.

The PCR buffer solution contains internal control DNA, and the same number of copies of internal control DNA are added respectively to the first and second containers. The PCR buffer solution further contains a PCR primer pair for amplifying the DNA in the specimen and the standard DNA, a PCR primer pair for amplifying the internal control DNA, an oligonucleotide fluorescence-labeled probe for detecting the amplified DNA in the specimen and the standard DNA, an oligonucleotide fluorescence-labeled probe for detecting the amplified internal control DNA, and DNA polymerase.

When the specimen is a sample containing RNA, the PCR buffer solution can further contain a reverse transcriptase and a reverse transcription reaction primer in order to generate cDNA by a reverse transcription reaction. The reverse transcriptase is an enzyme that generates single-stranded complementary DNA (cDNA) using RNA as a template, and is not particularly limited as long as it catalyzes the reverse transcription reaction, but RNA virus-derived RNA-dependent DNA polymerases such as avian myeloblastosis virus (AMV), moloney murine leukemia virus (M-MLV), human immunodeficiency virus (HIV), and the like, and variants of them can be used. As the reverse transcription reaction primer, a primer that specifically hybridizes to the sequence of the target RNA, an oligo (dT) primer, or a random primer can be used.

### (Internal Control DNA)

The internal control DNA has a sequence that does not hybridize with a PCR primer for amplifying the DNA in the specimen and the standard DNA and a probe for detecting the amplified DNA in the specimen and standard DNA in PCR Therefore, the internal control DNA is amplified and detected independently of the DNA in the specimen and the standard DNA. Therefore, the sequence of the internal control DNA is changed according to the sequence of DNA to be amplified in the specimen, and preferably a sequence that does not exist in nature is artificially synthesized. The chain length of the internal control DNA is preferably approximately 50 to 200 bp, and more preferably 80 to 120 bp for improving amplification efficiency. The GC-content of the internal control DNA is preferably approximately 40 to 60% in order to avoid a decrease in amplification efficiency. “Approximately” in the chain length and in the GC-content means that the chain length may be outside the range of 50 to 200 bp and the GC-content may be outside the range of 40 to 60% if the amplification efficiency is not reduced.

### (PCR Primer Pair for Amplifying DNA)

The PCR primer pair for amplifying DNA in the specimen and standard DNA is an oligonucleotide (forward and reverse) that hybridizes to a target base sequence under a stringent condition. The PCR primer pair for amplifying the internal control DNA is an oligonucleotide (forward and reverse) that hybridizes to a predetermined base sequence of the internal control DNA under a stringent condition. The stringent condition refers to a condition in which binding between a template DNA and a primer is specific in annealing in PCR, which is a step in which the primer is bound to the template DNA. The PCR primer pair for amplifying DNA in the specimen and standard DNA does not hybridize to the internal control DNA, and the PCR primer pair for amplifying the internal control DNA does not hybridize to the DNA in the specimen and the standard DNA. These primers preferably have a base length of 15 to 30 bases. It is necessary to design the base sequence of the primer to be used so that the amplification of the target gene region by each PCR primer pair proceeds well in one PCR reaction system.

### (Oligonucleotide Fluorescence-Labeled Probe)

In the present invention, it is possible to detect a PCR product by real-time measurement. The real-time measurement of the PCR product is also referred to as real-time PCR method. In the present invention, an oligonucleotide fluorescence-labeled probe is used to detect a PCR product by fluorescence. The oligonucleotide fluorescence-labeled probe for detecting the DNA in the specimen and the standard DNA hybridizes to a PCR product amplified by the PCR primer pair for amplifying the DNA in the specimen and the standard DNA under a stringent condition, but does not hybridize to a PCR product amplified by the PCR primer pair for amplifying the internal control DNA. In addition, the oligonucleotide fluorescence-labeled probe for detecting internal control DNA hybridizes to a PCR product amplified by the PCR primer pair for amplifying the internal control DNA under a stringent condition, but does not hybridize to a PCR product amplified by the PCR primer pair for amplifying the DNA in the specimen and the standard DNA. The stringent condition refers to a condition in which a specific hybrid is formed during annealing between a nucleic acid fragment amplified by PCR and an oligonucleotide fluorescence-labeled probe, and a non-specific hybrid is not formed. The oligonucleotide fluorescence-labeled probe used in the present invention preferably has a base length of 15 to 25 bases.

Examples of the fluorescence-labeled probe include a hydrolysis probe, a molecular beacon, and a cycling probe, but are not limited thereto. The hydrolysis probe is an oligonucleotide in which the 5′ end is modified with a fluorescent dye and the 3′ end is modified with a quencher substance. The hydrolysis probe specifically hybridizes to a template DNA in annealing of PCR, but a quencher is present on the hydrolysis probe, and therefore, generation of fluorescence is suppressed even when irradiated with excitation light. In the subsequent extension reaction step, when the hydrolysis probe hybridized to the template DNA is decomposed by the 5′→3′ exonuclease activity of Taq DNA polymerase, the fluorescent dye is released from the probe, and the suppression of generation of fluorescence by the quencher is released to emit fluorescence. It is possible to measure the production amount of the amplification product by measuring the fluorescence intensity. Examples of the fluorescent dye include FAM (6-carboxyfluorescein), ROX (6-carboxy-X-rhodamine), Cy3 and Cy5 (Cyanine-based dyes), HEX (4,7,2′,4′,5′,7′-hexachloro-6-carboxyfluorescein), and the like, but are not limited thereto. Examples of the quencher include TAMRA (registered trademark), BHQ (Black Hole Quencher, registered trademark) 1, BHQ2, MGB-Eclipse (registered trademark), DABCYL, and the like, but are not limited thereto.

The oligonucleotide fluorescence-labeled probe for detecting the DNA in the specimen and the standard DNA and the oligonucleotide fluorescence-labeled probe for detecting the internal control DNA are respectively bound to fluorescent dyes that are different from each other. Since the fluorescent dyes bound to the respective probes are different from each other, it is possible to separately measure PCR products obtained by the PCR primer pair for amplifying DNA in the specimen and the standard DNA and by the PCR primer pair for amplifying the internal control DNA. Combinations of fluorescent dyes that are different from each other are not particularly limited as long as they have different fluorescence characteristics and do not interfere with each other in fluorescence measurement.

### (DNA Polymerase)

The DNA polymerase is a thermostable DNA polymerase derived from thermophilic bacteria, and Taq, Tth, KOD, Pfu, and variants thereof can be used, but are not limited thereto. To avoid non-specific amplification by the DNA polymerase, a hot-start DNA polymerase may be used. Examples of the hot-start DNA polymerase include BIOTAQ (registered trademark) hot-start DNA polymerase. Examples of the hot-start DNA polymerase include a DNA polymerase to which an anti-DNA polymerase antibody is bound, and a DNA polymerase obtained by heat-sensitive chemical modification of an enzyme active site, and both can be used in the present invention.

### [Preparation of PCR Reaction Solution]

A PCR reaction solution for a specimen is adjusted by adding, to a first container, a specimen, internal control DNA, a PCR primer pair for amplifying the DNA in the specimen, a PCR primer pair for amplifying the internal control DNA, an oligonucleotide fluorescence-labeled probe for detecting the DNA in the specimen, an oligonucleotide fluorescence-labeled probe for detecting the internal control DNA, and a PCR buffer solution containing a DNA polymerase.

The specimen added to the first container is preferably mixed with the PCR buffer solution containing a surfactant in order to release DNA or RNA contained in the sample into the PCR reaction solution. As the surfactant contained in the PCR buffer solution, an anionic surfactant, a cationic surfactant, an amphoteric surfactant, or a nonionic surfactant can be selected, and a nonionic surfactant is preferable. Nonionic surfactants include, but are not limited to, Tween (registered trademark) 20 (polyoxyethylene sorbitan monolaurate), Tween 80 (polyoxyethylene sorbitan monooleate), Triton (registered trademark) X-100 (polyethylene glycol mono-4 octylphenyl ether), Nonidet (registered trademark) P-40 (octylphenyl-polyethylene glycol, NP-40), Brij (registered trademark)-35 (polyoxyethylene lauryl ether), and the like. The concentration of the surfactant to be used is preferably 0.05 to 5% (w/v) at the time of mixing with the specimen. The PCR buffer solution may further contain proteinase K. The proteinase K has an action of inactivating DNA and RNA degrading enzymes, and is preferably 100 to 300 μg/mL when mixed with the specimen.

In order to release RNA from a specimen containing an RNA virus such as a coronavirus, the specimen may be mixed with a specimen treatment solution containing sodium hydroxide as a main component and incubated at normal temperature to 95° C., and preferably at 80 to 95° C., for 3 minutes to 5 minutes. It is to be noted that the normal temperature is usually around 25° C. The specimen treatment solution may contain, other than sodium hydroxide, a metal chelating agent such as glycol ether diamine tetraacetic acid or the like and/or a reducing agent such as dithiothreitol or the like for the purpose of efficiently performing the RT (reverse transcription)-PCR treatment and enhancing the measurement accuracy. The specimen subjected to the incubation treatment with the specimen treatment solution is mixed with the PCR buffer solution, and thus is allowed to be subjected to the RT-PCR method.

A PCR reaction solution for preparing a calibration curve is adjusted by adding, to a second container, standard DNA, internal control DNA, a PCR primer pair for amplifying the standard DNA, a PCR primer pair for amplifying the internal control DNA, an oligonucleotide fluorescence-labeled probe for detecting the standard DNA, an oligonucleotide fluorescence-labeled probe for detecting the internal control DNA, and a PCR buffer solution containing a DNA polymerase. The internal control DNA added to the second container has the same number of copies as the internal control DNA added to the first container. The PCR primer pair for amplifying the DNA in the specimen added to the first container and the PCR primer pair for amplifying the standard DNA added to the second container are preferably the same, but may be different. Similarly, it is preferable that the oligonucleotide fluorescence-labeled probe for detecting the DNA in the specimen added to the first container and the oligonucleotide fluorescence-labeled probe for detecting the standard DNA added to the second container are the same, but they may be different.

Since the second container contains a known amount of serially diluted standard DNA, a plurality of second containers are prepared. A known amount of serially diluted standard DNA is used to prepare a calibration curve. The same operation as that applied to the first container is applied to the second container except that standard DNA is added instead of the specimen.

### [Measurement of Ct Value]

In the real-time measurement of the PCR product, the amplification curve of the PCR product is monitored using a fluorescent filter corresponding to the fluorescent dye used. The amplification curve can be created by plotting the fluorescence intensity against the number of PCR cycles. The Ct value (threshold cycle) is calculated as, for example, the number of PCR cycles corresponding to a point at which the threshold set in the rising region of the amplification curve intersects the amplification curve. From the real-time PCR method for the dilution series of the known amount of the standard DNA contained in the second container, the Ct value corresponding to the initial concentration of standard DNA at each point of the dilution series is calculated, and a calibration curve for quantifying DNA in the specimen can be obtained. The calibration curve plot is usually created with the Ct value for the standard DNA on the horizontal axis and the initial concentration of the standard DNA on the vertical axis. The DNA in the specimen can be quantified by applying the Ct value obtained for the specimen to the calibration curve.

Since the same number of copies of the internal control DNA is respectively added to the first container containing the specimen and the second container containing the standard DNA, the production amount of the PCR product of the internal control DNA is theoretically the same in each container and gives the same Ct value. In this case, there is no difference in signal values obtained from the real-time PCR method of each container. However, when a positive or negative error occurs in the signal value obtained from the real-time PCR method due to a composition difference of reaction solutions between a PCR reaction solution for a specimen and a PCR reaction solution for preparing a calibration curve and/or a difference between containers (well/tube) in a PCR process, it is difficult to accurately quantify DNA in the specimen. Since the error is a difference in the Ct value for the internal control DNA in the first container (for the specimen) and the second container (for the calibration curve), the Ct value for the DNA in the specimen contained in the first container can be corrected on the basis of the difference. The DNA in the specimen can be more accurately quantified by applying the corrected Ct value to the calibration curve.

For example, the correction value (Ctcv) of the Ct value (Ct) for DNA in the specimen contained in the first container can be calculated by the following formula.

Ct value for internal control DNA contained in first container (for specimen); Ct1

Ct value for internal control DNA contained in second container (for calibration curve); Ct2

$$Ctcv = Ct 2 / Ct 1 \times Ct$$

Ct1, Ct2, and Ct may each be an average value of values obtained from a plurality of containers.

### [Kit]

In a kit of the present invention, an internal control DNA, a PCR primer pair for amplifying DNA in a specimen and standard DNA, a PCR primer pair for amplifying internal control DNA, an oligonucleotide fluorescence-labeled probe, a DNA polymerase, and a PCR buffer solution may be each independently stored in different containers. For convenience of operation, an internal control DNA, a PCR primer pair for amplifying DNA in a specimen and standard DNA, a PCR primer pair for amplifying internal control DNA, an oligonucleotide fluorescence-labeled probe, a DNA polymerase, and a PCR buffer solution may be mixed in predetermined amounts, and stored in one container. Furthermore, depending on the intended use of the kit, the kit components can also be separately stored in 2 to 4 containers.

1. A method for quantifying DNA in a specimen, the method comprising:
a step of performing PCR by adding, to a first container containing a specimen and a second container containing a known amount of standard DNA, respectively a same number of copies of internal control DNA, a PCR primer pair for amplifying the DNA in the specimen and the standard DNA, a PCR primer pair for amplifying the internal control DNA, an oligonucleotide fluorescence-labeled probe for detecting the DNA in the specimen and the standard DNA, an oligonucleotide fluorescence-labeled probe for detecting the internal control DNA, and a PCR buffer solution containing a DNA polymerase;
a step of measuring a difference between Ct values in the PCR by comparing Ct values for the internal control DNA contained in the first container and the second container;
a step of correcting a Ct value for the DNA in the specimen contained in the first container on a basis of the difference between the Ct values; and
a step of measuring an amount of the DNA in the specimen from a calibration curve prepared on a basis of the amount of the standard DNA contained in the second container and the corrected Ct value.

2. The method according to claim 1, wherein the specimen is a sample selected from the group consisting of an organism sample, an organism-derived sample, an environmental sample, and an environmental-derived sample.

3. The method according to claim 1, wherein the specimen is a sample selected from the group consisting of an excrement sample, an excrement-derived sample, a vomit sample, and a vomit-derived sample.

4. The method according to claim 1, wherein the specimen contains a pathogen.

5. The method according to claim 4, wherein the pathogen is a virus, a bacterium, a fungus, or a protozoan.

6. The method according to claim 1, wherein the internal control DNA has a chain length of 50 to 200 bp and a GC-content of 40 to 60%.

7. The method according to claim 1, wherein the PCR buffer solution contains a surfactant.

8. The method according to claim 7, wherein the surfactant is a nonionic surfactant.

9. The method according to claim 1, wherein the PCR buffer solution is a tris buffer solution containing KCl, MgCl₂, and dNTP mix (mixture consisting of dATP, dGTP, dCTP, and dTTP).

10. The method according to claim 1, wherein the PCR buffer solution contains a substance that is bound to PCR-inhibiting substances, i.e., a bio-derived negative charge substance adsorbed to DNA polymerase and a bio-derived positive charge substance adsorbed to DNA, and neutralizes a PCR inhibitory action of the negative charge substance and the positive charge substance.

11. A kit for quantifying DNA in a specimen, the kit comprising:
internal control DNA;
a PCR primer pair for amplifying the DNA in the specimen and standard DNA;
a PCR primer pair for amplifying the internal control DNA;
an oligonucleotide fluorescence-labeled probe for detecting the DNA in the specimen and the standard DNA;
an oligonucleotide fluorescence-labeled probe for detecting the internal control DNA;
a DNA polymerase; and
a PCR buffer solution.

12. The kit according to claim 11, wherein the internal control DNA has a chain length of 50 to 200 bp and a GC-content of 40 to 60%.

13. The kit according to claim 11, wherein the PCR buffer solution contains a surfactant.

14. The kit according to claim 13, wherein the surfactant is a nonionic surfactant.

15. The kit according to claim 11, wherein the PCR buffer solution is a tris buffer solution containing KCl, MgCl₂, and dNTP mix (mixture consisting of dATP, dGTP, dCTP, and dTTP).

16. The kit according to claim 11, wherein the PCR buffer solution contains a substance that is bound to PCR-inhibiting substances, i.e., a bio-derived negative charge substance adsorbed to DNA polymerase and a bio-derived positive charge substance adsorbed to DNA, and neutralizes a PCR inhibitory action of the negative charge substance and the positive charge substance.
